(19) United States
(12) Patent Application Publication
(10) Pub. No.: US 2009/0299181 A1
(43) Pub. Date: Dec. 3, 2009

(54) DIAGNOSTIC IMAGING APPARATUS AND ULTRASONIC DIAGNOSTIC APPARATUS

(76) Inventors: Mayumi Ito, Tokyo (JP); Hiroshi Hashimoto, Tokyo (JP); Sei Kato, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/473,151

(22) Filed: May 27, 2009

(30) Foreign Application Priority Data
May 28, 2008 (JP) 2008-139599

(51) Int. Cl. A61B 8/14

(52) U.S. Cl. 600/440

(57) ABSTRACT

A diagnostic imaging apparatus includes an image generation device for generating a plurality of images of a subject for display in a display device, the plurality of images including a first image generated for display at a first display magnification and other images generated for display at other display magnification different from the first display magnification, and a marking setting device which sets a first marking to indicate a region of interest on the first image and other markings on the other images, the other markings being displayed, based on the other display magnification, in a position corresponding to the position of the first marking set on the first image.

### CROSS REFERENCE TO RELATED APPLICATIONS

This application claims the benefit of Japanese Patent Application No. 2008-139599 filed May 28, 2008, which is hereby incorporated by reference in its entirety.

### BACKGROUND OF THE INVENTION

The embodiments described herein relate to a diagnostic imaging apparatus, ultrasonic diagnostic apparatus, and program for displaying an image of a subject and also displaying a marking on the image to indicate a region of interest on the image, for example, to allow the size of the region to be measured.

An ultrasonic diagnostic apparatus disclosed in Japanese Unexamined Patent Publication No. Hei 6(1994)-178773 is an example of a diagnostic imaging apparatus that displays a marking on an image of a subject. In the ultrasonic diagnostic apparatus, a pattern for measurement such as a trace pattern is set on an ultrasonic image of a subject as a marking indicating a region of interest, allowing the size of the region of interest to be measured. Such measurement using a measurement pattern can be made not only on a single ultrasonic image but also on multiple ultrasonic images to compare mutually corresponding multiple regions of the multiple ultrasonic images. For example, to compare the size of a tumor before treatment and that of the tumor after treatment or to compare the size of a heart in a diastole phase and that of the heart in a systole phase, measurement patterns are displayed on ultrasonic images showing the tumors before and after treatment or the hearts in diastole and systole phases such that the sizes of the measurement patterns correspond to the sizes of regions of interest of the ultrasonic images. The sizes of the tumors or hearts determined based on such measurement patterns can then be compared.

It is, however, not easy to intuitively or visually compare the measurements obtained by the above method. With the measurements, the shapes of the regions of interest cannot be accurately compared, either.

The inventors of the invention have earnestly studied displaying a marking that is displayed to indicate a region of interest on a first image of a subject also in corresponding positions of other images of the subject. In this regard, there are demands for being able to magnify such a first image showing a region of interest to be indicated by a marking so as to allow the marking to be set with increased accuracy. It is therefore necessary to take into account instances where a first image on which a marking indicating a region of interest is displayed and other images on which corresponding markings are to be displayed in corresponding positions are displayed at different display magnifications.

It is desirable that the problems described previously are solved.

### BRIEF DESCRIPTION OF THE INVENTION

A first aspect provides a diagnostic imaging apparatus that includes an image generation device for generating a plurality of images of a subject for display in a display device, the plurality of images including a first image generated for display at a first display magnification and other images generated for display at other display magnification different from the first display magnification; and a marking setting device which sets a first marking to indicate a region of interest on the first image and other markings on the other images, the other marking being displayed, based on the other display magnification, in a position corresponding to the position of the first marking set on the first image.

A second aspect provides the diagnostic imaging apparatus according to the first aspect, further including a position information input device which inputs position information on the markings. In the diagnostic imaging apparatus, the marking setting device sets the markings based on the position information inputted from the position information input device.

A third aspect provides the diagnostic imaging apparatus according to the second aspect, wherein the position information inputted from the position information input device includes position information on the first marking indicating the region of interest on the first image.

A fourth aspect provides the diagnostic imaging apparatus according to one of the first to third aspects, wherein the first display magnification is larger than the other display magnification.

A fifth aspect provides the diagnostic imaging apparatus according to one of the first to fourth aspects, further including a magnification input device for inputting the first and other display magnifications. In the diagnostic imaging apparatus, the image generation device generates the images based on the display magnifications inputted from the magnification input device.

A sixth aspect provides the diagnostic imaging apparatus according to one of the first to fifth aspects, further including a measurement device which measures, based on the first marking, an image portion in the region of interest on the first image.

A seventh aspect provides the diagnostic imaging apparatus according to one of the first to sixth aspects, wherein the markings on the first and other images are displayed in different display modes.

An eighth aspect provides the diagnostic imaging apparatus according to the seventh aspect, wherein the markings displayed on the first and other images mutually differ in color or line type.

A ninth aspect provides the diagnostic imaging apparatus according to one of the first to eighth aspects, wherein the plurality of images generated by the image generation device includes a real-time image based on data acquired in real time and a past image based on data acquired at a past time.

A tenth aspect provides the diagnostic imaging apparatus according to one of the first to eighth aspects, wherein the plurality of images generated by the image generation device includes past images based on data acquired at different past times.

An eleventh aspect provides the diagnostic imaging apparatus according to one of the first to tenth aspects, further including an editing unit for editing the markings displayed on the first and other images.

A twelfth aspect provides the diagnostic imaging apparatus according to the eleventh aspect, wherein, when the markings are edited by the editing unit, the measurement device performs measurement based on the edited markings.

A thirteenth aspect provides the diagnostic imaging apparatus according to one of the first to twelfth aspects, wherein the markings are circular, ellipsoidal, polygonal, or linear.

A fourteenth aspect provides the diagnostic imaging apparatus, wherein the markings are traces of positions specified by the position information input device.

A fifteenth aspect provides the diagnostic imaging apparatus according to one of the second to fourteenth aspects, wherein the position information input device inputs position information on a cursor displayed on the first image, and wherein the marking setting device sets the markings based on the position information on the cursor inputted from the position information input device.

A sixteenth aspect provides an ultrasonic diagnostic apparatus that includes an ultrasonic probe which transmits and receives an ultrasonic wave to and from a subject; an image generation device which generates, for display in a display device, a plurality of ultrasonic images of a subject, the plurality of ultrasonic images including a first ultrasonic image generated for display at a first display magnification and other ultrasonic images generated for display at other display magnification different from the first display magnification; and a marking setting device which sets a first marking to indicate a region of interest on the first ultrasonic image and other markings on the other ultrasonic images, the other marking being displayed, based on the other display magnification, in a position corresponding to the position of the first marking set on the first ultrasonic image.

A seventeenth aspect provides a program which causes a diagnostic imaging apparatus for displaying an image of a subject in a display device to execute: an image generation function for generating a plurality of images of a subject, for display in the display device, at different display magnifications; and a marking setting function for setting a first marking to indicate a region of interest on a first image included in the plurality of images and other markings on other images included in the plurality of images, the other markings being set for display, based on a display magnification for the other images, in a position corresponding to the position of the first marking set on the first image.

According to the first aspect, the first marking is displayed on the first image, and the other markings are displayed on the other images, the other markings being displayed, based on the other display magnification, in a position corresponding to the position of the first marking set on the first image. This makes it possible to easily and accurately compare the regions of interest on the first and other images. Even when the first and other images are displayed at mutually different display magnifications, the marking setting device can easily set the first and other markings taking into account the difference between the two display magnifications.

According to the second aspect, the markings can be set on the first and other images by inputting position information on the markings from the position information input device.

According to the third aspect, the markings can be set on the first and other images by inputting position information on the marking to indicate a region of interest on the first image from the position information input device.

According to the fourth aspect, the first display magnification is larger than the other display magnification. Therefore, when position information on the marking to indicate a region of interest on the first image is inputted, the region of interest can be easily and accurately indicated by the marking.

According to the fifth aspect, arbitrary display magnifications can be inputted for the first and other images from the magnification input device, and the images can be displayed at the arbitrary display magnifications.

According to the sixth aspect, the measurement device can measure the region of interest on the first image.

According to the seventh aspect, the markings on the first and other images are displayed in different display modes, so that the markings can be easily distinguished.

According to the eighth aspect, the markings displayed on the first and other images mutually differ in color or line type, i.e. displayed in different display modes, so that they can be easily distinguished.

According to the ninth aspect, it is possible to compare a region of interest on a real-time image and a region of interest on a past image.

According to the tenth aspect, it is possible to compare multiple regions of interest on multiple past images.

According to the eleventh aspect, the markings displayed on the first and other images can be edited by the editing unit.

According to the twelfth aspect, it is possible to perform measurement based on the markings having been edited.

According to the thirteenth aspect, the markings to indicate regions of interest are circular, circular, ellipsoidal, polygonal, or linear.

According to the fourteenth aspect, the markings to indicate regions of interest are traces of positions specified by the position information input device.

According to the fifteenth aspect, the markings can be set based on the cursor displayed on the first image.

According to the sixteenth aspect, an ultrasonic diagnostic apparatus having effects similar to those of the first aspect can be obtained.

According to the seventeenth aspect, effects similar to those of the first aspect can be obtained.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the configuration of an ultrasonic diagnostic apparatus according to an embodiment of the invention.

FIG. 2 is a block diagram showing, in detail, the configurations of the image display control device and the control device of the ultrasonic diagnostic apparatus shown in FIG. 1.

FIG. 3 is a diagram showing an example display of a marking.

FIG. 4 is a diagram showing images shown in the display device of the ultrasonic diagnostic apparatus shown in FIG. 1.

FIG. 5 is a diagram showing images shown in the display device of the ultrasonic diagnostic apparatus shown in FIG. 1.

FIG. 6 is a diagram showing images shown in the display device of the ultrasonic diagnostic apparatus shown in FIG. 1.

FIG. 7 is a diagram showing another example display of images in the display device.

### DETAILED DESCRIPTION OF THE INVENTION

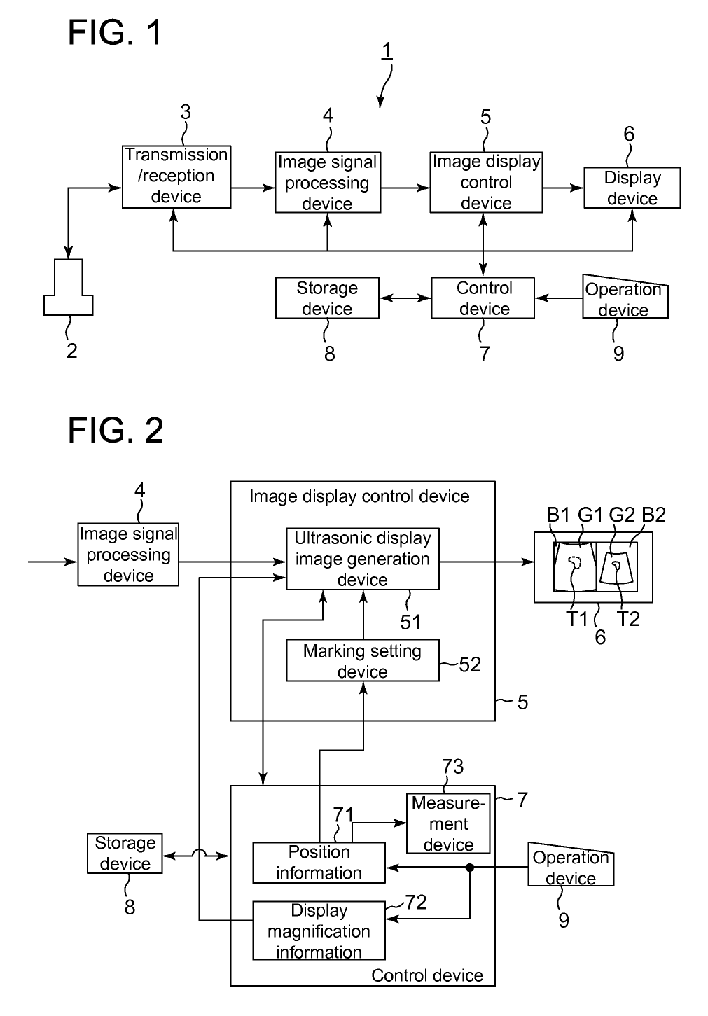

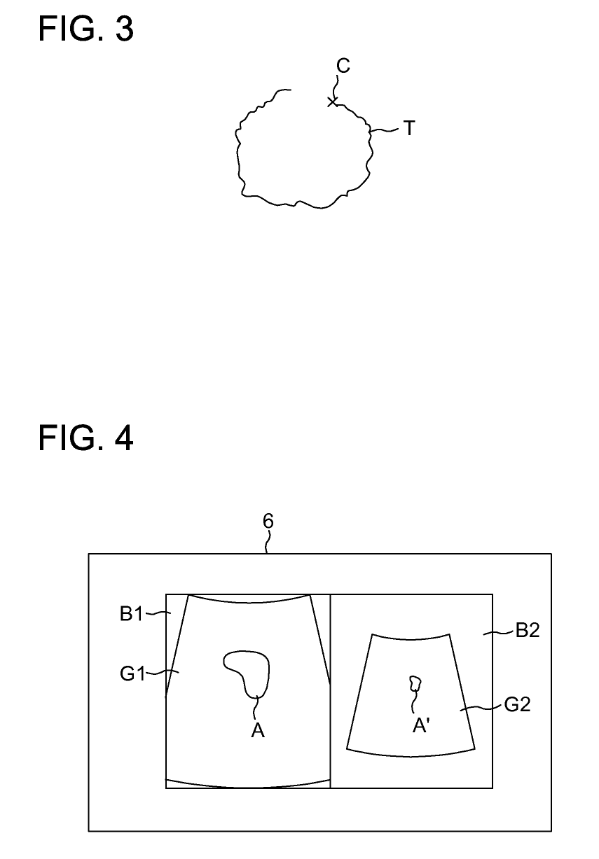

As an example embodiment of the invention, an ultrasonic diagnostic apparatus will be described in detail below with reference to drawings. FIG. 1 is a block diagram showing the configuration of an exemplary ultrasonic diagnostic apparatus according to an embodiment of the invention. FIG. 2 is a block diagram showing, in detail, the configurations of the image display control device and the control device of the ultrasonic diagnostic apparatus shown in FIG. 1. FIG. 3 is a diagram showing an example display of a marking. FIGS. 4 to 6 each shows example images shown in the display device of the ultrasonic diagnostic apparatus shown in FIG. 1.

The ultrasonic diagnostic apparatus 1 shown in FIG. 1 includes an ultrasonic probe 2, a transmission/reception device 3 which drives the ultrasonic probe 2 to scan an object surface and obtains a sound ray signal, for example, by subjecting an echo signal obtained using the ultrasonic probe 2 to phasing addition, an image signal processing device 4 which obtains an ultrasonic image signal, for example, by subjecting the sound ray signal outputted from the transmission/reception device 3 to logarithmic amplification and envelope demodulation, an image display control device 5 which generates, based on the ultrasonic image signal outputted from the image signal processing device 4 and other information, image data and displays an ultrasonic image in a display device 6 being described later, and the display device 6 for displaying an ultrasonic image.

The ultrasonic diagnostic apparatus 1 also includes a control device 7 which controls the apparatus as a whole, a storage device 8 which stores ultrasonic image data and programs, and an operation device 9 which is provided with a keyboard and a pointing device and through which the operator inputs instructions.

The image display control device 5 includes, as shown in FIG. 2, an ultrasonic display image generation device 51 which generates ultrasonic images of a subject to be displayed in the display device 6 and a marking setting device 52 which sets a marking for indicating a region of interest on an ultrasonic image generated by the ultrasonic display image generation device 51.

The ultrasonic display image generation device 51 is an example embodiment of the image generation device according to the invention. The ultrasonic display image generation device 51 generates ultrasonic images G1 and G2 to be displayed in display partitions B1 and B2, respectively, of the display device 6. It generates the ultrasonic images G1 and G2 based on ultrasonic image signals received from the image signal processing device 4 and ultrasonic image data stored in the storage device 8. The ultrasonic images G1 and G2 differ from each other in display magnification. Namely, the ultrasonic display image generation device 51 generates the ultrasonic images G1 and G2 magnified at predetermined display magnifications, respectively, based on display magnification information 72 inputted from the control device 7.

The ultrasonic images G1 and G2 are based on echo data obtained, at different times, from a same region of a subject. For example, the ultrasonic images G1 and G2 can be two images of a same region of a subject, one being a real-time image based on real-time echo data and the other being a past image based on past echo data. They may also be two past images of a same region of a subject generated based on echo data obtained at different times.

In the present example, a tumor A (see FIGS. 4 to 6) is indicated by the marking (i.e. a trace T being described later) as a region of interest on the ultrasonic image G1, and the size of the tumor A thus marked is measured. The ultrasonic image G1 is an example embodiment of the first image according to the invention. The ultrasonic image G2 is an example embodiment of the other image according to the invention.

The marking setting device 52 is an example embodiment of the marking setting device according to the invention. In the present embodiment, the marking setting device 52 displays a pattern for measurement, as the marking, used to measure the size of a region of interest. In the present embodiment, a trace T of a cursor C on an ultrasonic image is displayed as the pattern for measurement as shown in FIG. 3.

The marking setting device 52 sets a marking on each of the ultrasonic images G1 and G2. Namely, the marking setting device 52 sets a marking on the ultrasonic image G1 based on position information 71 inputted from the control device 7, and sets another marking, in the position corresponding to the marking position on the ultrasonic image G1, on the ultrasonic image G2 based on the display magnification predetermined for the ultrasonic image G2.

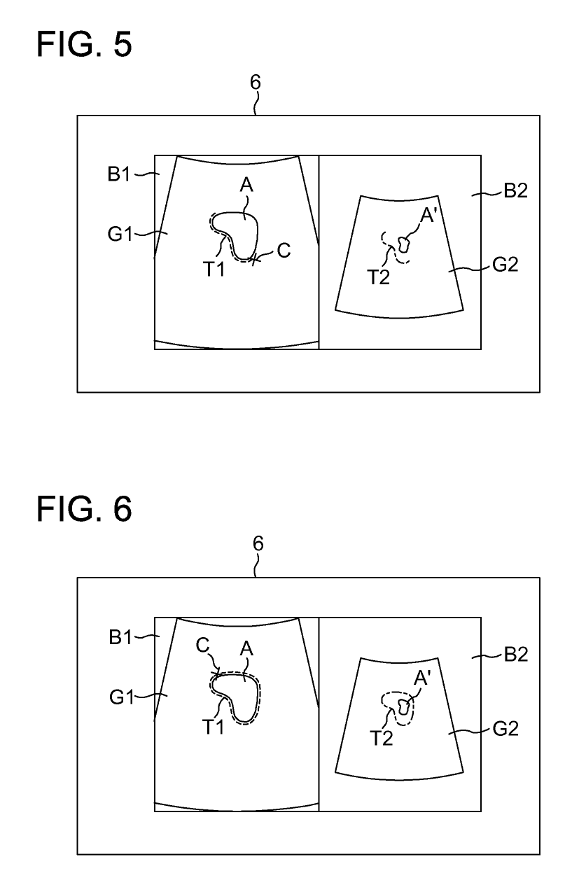

On the ultrasonic images G1 and G2, the markings are displayed in different display modes, that is, for example, in different line types and/or in different colors. The two markings displayed in mutually different line types or colors can be easily distinguished. In the present embodiment, trace T1 is displayed in broken line on the ultrasonic image G1, whereas trace T2 is displayed in dashed line on the ultrasonic image G2 (see FIGS. 5 and 6). The traces T1 and T2 may mutually differ in color.

The control device 7 includes, for example, a central processing unit (CPU) and executes, by executing control programs stored in the storage device 8, the functions of various devices included in the ultrasonic diagnostic apparatus 1. The image generation function and marking setting function according to the invention are also executed by the control device 7 by executing such control programs.

The control device 7 has the position information 71 based on which the markings are displayed. The position information 71 is generated from the position information on cursor C inputted from the pointing device (not shown) included in the operation device 9. Namely, the markings represent a trace T of the cursor C. The position information 71 represents position information inputted from a point on the trace T. The position information 71 is updated every time information is inputted from the pointing device, and outputted to the marking setting device 52 every time it is updated. The operation device 9 is an example embodiment of the position information input device according to the invention.

The operation device 9 enables the markings set on the ultrasonic images G1 and G2 to be edited, for example, by moving, rotating, or inverting them, or by changing their sizes and shapes. To be concrete, using the pointing device to place the cursor C on the marking displayed on the ultrasonic image G1 or G2 and then dragging the cursor C updates the position information on the marking, causing the marking to be moved or its size or shape to be changed. The operation device 9 is an example embodiment of the editing unit according to the invention.

The control device 7 has the display magnification information 72 for ultrasonic images. The display magnification information 72 is inputted from the keyboard (not shown) included in the operation device 9 and outputted to the ultrasonic display image generation device 51. The operation device 9 is an example embodiment of the magnification input device according to the invention.

The control device 7 is provided with a measurement device 73 that measures an image size based on the position information 71. To be concrete, it determines the size of the area inside the trace T. Though not shown in the attached drawings, the area size thus determined may be displayed in the display device 6.

When the position information 71 is updated, the measurement device 73 measures the image size again and updates the measurement.

The operation of the ultrasonic diagnostic apparatus 1 of the present embodiment will be described in the following. First, in accordance with instructions inputted by the operator, the ultrasonic display image generation device 51 displays, as shown in FIG. 4, ultrasonic images G1 and G2 in the display partitions B1 and B2 of the display device 6. In the present embodiment, the ultrasonic image G1 is a past image stored in the storage device 8, and the ultrasonic image G2 is a real-time image based on data acquired in real time. In the present example, the ultrasonic image G1 is an image showing the tumor A before being treated, and the ultrasonic image G2 is an image showing the tumor A′, i.e. the tumor A after being treated and reduced in size.

The ultrasonic display image generation device 51 receives image data read by the control device 7 from the storage device 8, generates the ultrasonic image G1, and displays the image in the display partition B1. The ultrasonic display image generation device 51 also generates the ultrasonic image G2 based on an ultrasonic image signal inputted from the image signal processing device 4 and displays the image in the display partition B2.

The ultrasonic images G1 and G2 are displayed at predetermined display magnifications, respectively. The display magnifications for the ultrasonic images G1 and G2 may be inputted from the operation device 9 immediately before the ultrasonic images are displayed, or they may be stored ones having been inputted from the operation device 9. In the present embodiment, the ultrasonic image G1 is displayed at a higher display magnification than the ultrasonic image G2. Namely, the ultrasonic image G1 is displayed more enlarged than the ultrasonic image G2.

Next, the operator is to display, from the operation device 9, the markings on the ultrasonic images G1 and G2. Namely, when the operator moves, using the pointing device included in the operation 9, the cursor C shown on the ultrasonic image G1, as shown in FIG. 5, along the contour of the tumor A also shown on the ultrasonic image G1, the trace T1 is displayed. As the cursor C is moved on the ultrasonic image G1 causing the trace T1 to be displayed, the trace T2 is displayed in the corresponding position on the ultrasonic image G2.

After displaying the trace T1 enclosing the tumor A as shown in FIG. 6, the operator inputs a measurement command from the operation device 9 to measure the tumor area enclosed in the trace T1.

According to the ultrasonic diagnostic apparatus 1 of the present embodiment described above, the trace T1 is displayed, as a marking, on the ultrasonic image G1 and, at the same time, the trace T2 is displayed in the corresponding position on the ultrasonic image G2 at the display magnification predetermined for the ultrasonic image G2. It is therefore possible to easily and accurately compare the sizes of the tumors A and A′ on the ultrasonic image G2. Even when the display magnifications for the ultrasonic images G1 and G2 are different, the marking setting device 52 makes it possible to set the trace T2 with ease based on the display magnification for the ultrasonic image G2.

With the display magnification for the ultrasonic image G1 larger than that for the ultrasonic image G2, the ultrasonic image G1 is displayed more enlarged than the ultrasonic image G2. This facilitates moving the cursor C along the contour of the tumor A shown on the ultrasonic image G1. Since the tumor A is accurately marked with the trace T1, the tumor size can be measured accurately.

Even though the invention has been described based on the above embodiment, various modifications can be made to the invention without departing from its scope. For example, the markings may be circular, ellipsoidal, polygonal, or linear. The invention can be applied, not only to ultrasonic diagnostic apparatuses, but also to other diagnostic imaging apparatuses such as X-ray computed tomography (CT) scanners and magnetic resonance imaging (MRI) apparatuses.

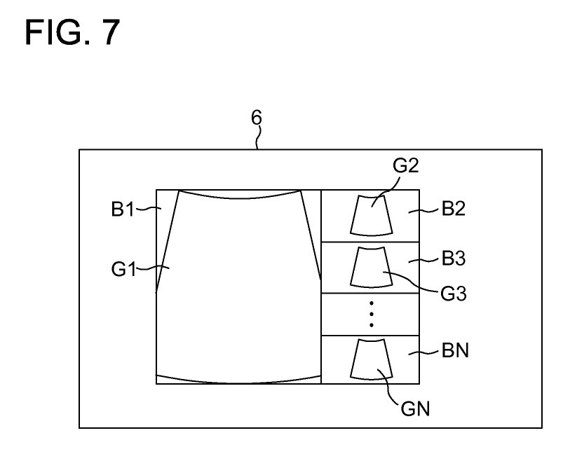

Even though, in the above embodiment, the ultrasonic image G2 is the only image other than the ultrasonic image G1, there may be multiple images other than the ultrasonic image G1. Namely, the ultrasonic display image generation device 51 may generate, in addition to the ultrasonic image G1 for display in the display partition B1, multiple ultrasonic images GK ((K=2 to N) where N is 3 or a larger natural number) to be displayed in multiple display partitions BK ((K=2 to N) where N is 3 or a larger natural number) as shown in FIG. 7. In such a case, on each of the ultrasonic images G2, G3, - - - , GN displayed in the display partitions B2, B3, - - - , BN, a marking is displayed (not shown) in the position corresponding to the position of the marking (not shown in FIG. 7) shown on the ultrasonic image G1 and at the display magnification predetermined for each of the ultrasonic images G2, G3, - - - , GN.

The ultrasonic images G2, G3, - - - , GN may be displayed either at a same display magnification or at mutually different display magnifications. In the example shown in FIG. 7, the ultrasonic images G2, G3, - - - , GN are displayed at a same display magnification different from the display magnification at which the ultrasonic image G1 is displayed.

Many widely different embodiments of the invention may be configured without departing from the spirit and the scope of the present invention. It should be understood that the present invention is not limited to the specific embodiments described in the specification, except as defined in the appended claims.

1. A diagnostic imaging apparatus comprising:
an image generation device configured to generate a plurality of images of a subject for display in a display device, the plurality of images including a first image generated for display at a first display magnification and other images generated for display at other display magnification different from the first display magnification; and
a marking setting device configured to set a plurality of markings including a first marking to indicate a region of interest on the first image and other markings on the other images, the other markings being displayed, based on the other display magnification, in a position corresponding to the position of the first marking set on the first image.

2. The diagnostic imaging apparatus according to claim 1, further comprising a position information input device configured to input position information on the plurality of markings, wherein the marking setting device is configured to set the markings based on the position information inputted from the position information input device.

3. The diagnostic imaging apparatus according to claim 2, wherein the position information inputted from the position information input device includes position information on the first marking indicating the region of interest on the first image.

4. The diagnostic imaging apparatus according to claim 1, wherein the first display magnification is larger than the other display magnification.

5. The diagnostic imaging apparatus according to claim 1, further comprising a magnification input device configured to input the first display magnification and the other display magnifications, wherein the image generation device is configured to generate the plurality of images based on the display magnifications inputted from the magnification input device.

6. The diagnostic imaging apparatus according to claim 1, further comprising a measurement device configured to measure, based on the first marking, an image portion in the region of interest on the first image.

7. The diagnostic imaging apparatus according to claim 1, wherein the markings on the first and the other images are displayed in different display modes.

8. The diagnostic imaging apparatus according to claim 7, wherein the markings displayed on the first and other images mutually differ in one of a color and a line type.

9. The diagnostic imaging apparatus according to claim 1, wherein the plurality of images generated by the image generation device includes a real-time image based on data acquired in real time and a past image based on data acquired at a past time.

10. The diagnostic imaging apparatus according to claim 1, wherein the plurality of images generated by the image generation device includes past images based on data acquired at different past times.

11. The diagnostic imaging apparatus according to claim 1, further comprising an editing unit configured to edit the first marking displayed on the first image and the other markings displayed on the other images.

12. The diagnostic imaging apparatus according to claim 11, wherein, when the markings are edited by the editing unit, the measurement device is configured to perform a measurement based on the edited markings.

13. The diagnostic imaging apparatus according to claim 1, wherein the markings are one of circular, ellipsoidal, polygonal, and linear.

14. The diagnostic imaging apparatus according to claim 2, wherein the markings are traces of positions specified by the position information input device.

15. The diagnostic imaging apparatus according to claim 2,
wherein the position information input device is configured to input position information on a cursor displayed on the first image, and
wherein the marking setting device is configured to set the plurality of markings based on the position information on the cursor inputted from the position information input device.

16. An ultrasonic diagnostic apparatus comprising:
an ultrasonic probe configured to transmit and receive an ultrasonic wave to and from a subject;
an image generation device configured to generate, for display in a display device, a plurality of ultrasonic images of a subject, the plurality of ultrasonic images including a first ultrasonic image generated for display at a first display magnification and other ultrasonic images generated for display at other display magnification different from the first display magnification; and
a marking setting device configured to set a plurality of markings including a first marking to indicate a region of interest on the first ultrasonic image and other markings on the other ultrasonic images, the other markings being displayed, based on the other display magnification, in a position corresponding to the position of the first marking set on the first ultrasonic image.

17. The diagnostic imaging apparatus according to claim 16, further comprising a magnification input device configured to input the first display magnification and the other display magnifications, wherein the image generation device is configured to generate the plurality of images based on the display magnifications inputted from the magnification input device.

18. The diagnostic imaging apparatus according to claim 16, further comprising a measurement device configured to measure, based on the first marking, an image portion in the region of interest on the first image.

19. The diagnostic imaging apparatus according to claim 16, wherein the plurality of images generated by the image generation device includes a real-time image based on data acquired in real time and a past image based on data acquired at a past time.

20. The diagnostic imaging apparatus according to claim 16, further comprising an editing unit configured to edit the first marking displayed on the first image and the other markings displayed on the other images.
